(19) United States
(12) Patent Application Publication
(10) Pub. No.: US 2024/0019442 A1
(43) Pub. Date: Jan. 18, 2024

(54) BIOSENSOR FOR DETECTING STRUCTURAL CHANGES IN HUMAN CARDIAC MUSCLE PROTEIN

(71) Applicant: ARIZONA BOARD OF REGENTS ON BEHALF OF THE UNIVERSITY OF ARIZONA, Tucson, AZ (US)

(72) Inventors: Brett Colson, Tucson, AZ (US); Rhye-Samuel Kanassatega, Tucson, AZ (US)

(21) Appl. No.: 18/044,344

(22) PCT Filed: Sep. 10, 2021
(86) PCT No.: PCT/US21/49836

§ 371 Date: Mar. 7, 2023

Related U.S. Application Data
(63) Continuation-in-part of application No. PCT/US21/14142, filed on Jan. 20, 2021.
(60) Provisional application No. 63/076,735, filed on Sep. 10, 2020.
(60) Provisional application No. 62/963,298, filed on Jan. 20, 2020.

(51) Int. Cl. G01N 33/58

(52) U.S. Cl. CPC G01N 33/582; G01N 2333/4712

(57) ABSTRACT

Cardiac Myosin Binding Protein-C (MyBP-C) is a thick filament-associated protein of the sarcomere and a potential therapeutic target for treating heart failure. The present invention utilizes the MyBP-C N-terminal domains C0 through C2 (C0-C2) to create a fluorescent protein biosensor suitable for time-resolved fluorescence energy transfer (TR-FRET). The dynamic range and precision of the phosphorylation-mediated changes of the fluorescent protein biosensor demonstrate excellent assay functionality for the purposes of high-throughput screening and enabling scientific discovery for understanding MyBP-C in health and disease. These findings provide new molecular insight into the regulatory role of MyBP-C in contractility and demonstrate the potential of C0-C2 biosensors for structure-based screening of compounds in search of MyBP-C-targeted therapies for heart failure.

### CROSS-REFERENCES TO RELATED APPLICATIONS

This application claims benefit of U.S. Provisional Application No. 63/076,735 filed Sep. 10, 2020, the specification(s) of which is/are incorporated herein in their entirety by reference.

This application is a continuation-in-part and claims benefit of PCT Application No. PCT/US21/14142, filed Jan. 20, 2021, the specification(s) of which is/are incorporated herein in their entirety by reference.

### STATEMENT REGARDING FEDERALLY SPONSORED RESEARCH OR DEVELOPMENT

This invention was made with government support under Grant No. R01 HL141564, awarded by National Institutes of Health. The government has certain rights in the invention.

### FIELD OF THE INVENTION

The present invention relates to protein biosensors for drug discovery, in particular, to a high-throughput assay utilizing time-resolved fluorescence energy transfer (TR-FRET) to detect phosphorylation-mediated structural changes.

### BACKGROUND OF THE INVENTION

Cardiac myosin-binding protein C (cMyBP-C) is the most commonly mutated gene associated with hypertrophic cardiomyopathy (HCM), which can also lead to heart failure (HF). HF, most commonly caused by coronary artery disease, high blood pressure, and previous heart attack, is frequently associated with dysregulated cMyBP-C phosphorylation. Thus, cMyBP-C is a prime target for compounds that upon binding restore its function to treat HF and HCM. cMyBP-C is a sarcomeric protein found in 9 axial stripes, spaced ˜43 nm apart due to interactions with titin and myosin in the C-zone of the A-band (FIG. 1A). Structurally, cMyBP-C is a ˜140 kDa protein consisting of 8 Igl-like and 3 fibronectin-3 domains, referred to as C0-C10 (FIG. 1B). The C-terminal portion (C8-C10) is bound to the thick myosin-containing filaments due to strong interactions with myosin and weaker interactions with titin. The N-terminal portion (C0-C2) (FIG. 1C) extends away from the backbone towards potential interactions with myosin subfragment-2 (S2) and the regulatory light chain (RLC) of myosin, actin, and tropomyosin (Tm) of the thin filament. These interactions with the thick and thin filaments are thought to regulate contractile function.

The activation of the beta-adrenergic signaling pathway leads to phosphorylation of sarcomeric proteins to increase cardiac muscle contraction and relaxation kinetics. The phosphorylation of cMyBP-C contributes to proper diastolic function and responsiveness to beta-adrenergic activation, leading to increased contractility and cardiac output by regulating actin-myosin interactions. At low Ca²⁺ levels, cMyBP-C activates thin filaments by interactions with actin-Tm, but at high Ca²⁺, cMyBP-C plays an inhibitory role on the actin-myosin interaction. Upon beta-adrenergic stimulation, protein kinase A (PKA) phosphorylates cMyBP-C in the M-domain (connecting C1 to C2, FIG. 1B-D), which results in a marked reduction in the binding of N-terminal cMyBP-C fragments to actin and myosin filaments. This phosphorylation of cMyBP-C, as well as cardiac troponin-I (cTnl), and titin, modulates cardiac performance via beta-adrenergic signaling on a beat-to-beat basis in healthy myocardium.

In studies of healthy human donor hearts, cMyBP-C is highly phosphorylated on four serines in the M-domain (FIGS. 1B, 1C, and 1E). In contrast, patients diagnosed with heart failure (HF) and comorbidities that lead to HF (i.e. chronic atrial fibrillation and hypertrophic cardiomyopathy) show ˜40-75% total reduction in cMyBP-C phosphorylation. In HF, the distribution of mono-, di-, tris-, and tetra-phosphorylated cMyBP-C shifts from tris- and tetra-phosphorylated molecules (constituting ˜65%) in healthy hearts to predominantly unphosphorylated and monophosphorylated cMyBP-C molecules (constituting ˜88% in total). Mice expressing phosphomimetic charge substitutions at each phosphoserine in cMyBP-C display cardioprotective phenotypes evident by preserved cardiac morphology, enhanced myocardial relaxation, and attenuated age-related cardiac dysfunction. Moreover, in a rat HF model, reduced cMyBP-C phosphorylation coincided with decompensation, further supporting cMyBP-C phosphorylation as a target for new therapy in diseased myocardium. Therefore, new methods and systems to study compounds that affect cMyBP-C phosphorylation and lead to HF are needed.

### BRIEF SUMMARY OF THE INVENTION

It is an objective of the present invention to provide compositions and methods that allow for the quantification of phosphorylation-mediated structural changes of a protein in a high-throughput manner, as specified in the independent claims. Embodiments of the invention are given in the dependent claims. Embodiments of the present invention can be freely combined with each other if they are not mutually exclusive.

Cardiac Myosin Binding Protein-C (MyBP-C) is a thick filament-associated protein of the sarcomere and a potential therapeutic target for treating contractile dysfunction in heart failure. Mimicking the structural dynamics of phosphorylated cMyBP-C, which is suggested to be cardioprotective, by small-molecule drug binding could lead to therapies that modulate cMyBP-C conformational states to improve contractility.

Human cMyBP-C N-terminal fragment C0-C2 is ˜50 kDa and contains two flexible linker regions, the proline/alanine-rich linker (P/A) connecting C0 to C1 and the motif domain (M-domain) (FIG. 1C). The M-domain is mostly intrinsically disordered, expressing four serines phosphorylated by PKA at positions 275, 284, 304, and 311. However, the C-terminal portion of the M-domain contains a structured three-helix bundle at residues 322-357 termed the tri-helix bundle.

The present invention has developed a human cMyBP-C biosensor capable of detecting intramolecular structural changes due to phosphorylation. Site-directed mutagenesis of cMyBP-C N-terminal domains C0 through C2 (C0-C2) substituted endogenous cysteines and introduced new cysteines for time-resolved fluorescence resonance energy transfer (TR-FRET)-based examination of C0-C2 structure. A cysteine pair (di-Cys C0-C2) was identified that upon donor-acceptor labeling reports phospho-sensitive structural changes between the C1 domain and the tri-helix bundle of the M-domain which links C1 to C2. Phosphorylation reduced FRET efficiency by ˜17%, corresponding to a ˜9% increase in the distance between probes and a ˜24% increase in disorder between them. The magnitude and precision of phosphorylation-mediated TR-FRET changes as quantified by the Z′-factor demonstrate the assay's potential for high-throughput structure-based screening of compounds for cMyBP-C-targeted therapies to improve cardiac performance in the failing human heart. Additionally, by probing C1's spatial positioning relative to the tri-helix bundle, these findings provide new molecular insight into the structural dynamics of phosphoregulation in cMyBP-C.

The present invention features a method of using time-resolved fluorescence energy transfer (TR-FRET) and a fluorescent protein biosensor to quantitate phosphorylation-mediated structural changes in a solution. In some embodiments, the method comprises labeling (operably connecting) a protein with two fluorescent probes to generate a fluorescent protein biosensor suitable for TR-FRET. In other embodiments, the method comprises measuring FRET efficiency when structural changes in the fluorescent protein biosensor occur, wherein FRET efficiency is the proportion of the donor molecules that have transferred excitation state energy to the acceptor molecules. In further embodiments, the method comprises quantitating protein structural changes including phosphorylation using the measured FRET efficiency.

The present invention may also feature a method of using time-resolved fluorescence energy transfer (TR-FRET) to quantitate myosin binding protein-C (MyBP-C) phosphorylation-mediated structural changes in solution. In some embodiments, the method comprises labeling (operably connecting) a MyBP-C with two fluorescent probes (or one fluorescent probe and one non-fluorescent quencher) for TR-FRET. In other embodiments, the method comprises measuring FRET efficiency when structural changes in the MyBP-C occur, wherein FRET efficiency is the proportion of the donor molecules that have transferred excitation state energy to the acceptor molecules. In further embodiments, the method comprises quantitating protein structural changes including phosphorylation using the measured FRET efficiency.

Additionally, the present invention features a fluorescent biosensor protein suitable for time-resolved fluorescence energy transfer (TR-FRET). In some embodiments, the fluorescent biosensor protein comprises two fluorescent probes operably connected to the protein. In other embodiments, the fluorescent biosensor protein is capable of quantitating phosphorylation-mediated structural changes in solution.

The present invention may also feature a myosin-binding protein C (MyBP-C) fluorescent biosensor suitable for time-resolved fluorescence energy transfer (TR-FRET). In some embodiments, the MyBP-C fluorescent biosensor comprises two fluorescent probes operably connected to the protein. In other embodiments, the MyBP-C fluorescent biosensor protein is capable of quantitating phosphorylation-mediated structural changes in solution.

One of the unique and inventive technical features of the present invention is the site-directed probes in the myosin binding protein C (MyBP-C) to create a fluorescent biosensor protein. Without wishing to limit the invention to any theory or mechanism, it is believed that the technical feature of the present invention advantageously provides for the capability of detecting structural changes due to phosphorylation using time-resolved fluorescence energy transfer (TR-FRET). Additionally, mimicking the structural state of phosphorylated MyBP-C by small-molecule drugs could lead to therapy that optimizes MyBP-C function in the failing heart. None of the presently known prior references or work has the unique inventive technical feature of the present invention.

Furthermore, the prior references teach away from the present invention. For example, mice and humans have distinct sequences in N-terminal cMyBP-C regions important for function (binding and phosphorylation). While both mouse and human both have 5 cysteines within the MyBP-C, attempts to label the cysteines produced distinct products whereas only one cysteine was labeled in the mouse version of the protein, greater than one cysteine was labeled in the human version of the protein. Therefore, insights gained using mouse MyBP-C, do not necessarily correlate to human MyBP-C.

Furthermore, the inventive technical features of the present invention contributed to a surprising result. For example, the probes most sensitive to PKA-mediated phosphorylation of mouse cMyBP-C caused decreased distance and disorder, whereas effects of phosphorylation on human cMyBP-C were not detected at these sites, and at other sites phosphorylation of human cMyBP-C, surprisingly, caused increased distance and disorder. In another example, thiol-reactive dye labeling of human MyBP-C was optimized to maximize labeling of cysteines with negligible labeling non-specific residues by reducing pH to 6.5 (mildly acidic), which ensures accuracy of the structural measurements in that the detectable FRET signals come only from the probes attached to the two cysteines.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE SEVERAL VIEWS OF THE DRAWING(S)

The features and advantages of the present invention will become apparent from a consideration of the following detailed description presented in connection with the accompanying drawings in which:

FIGS. 1A, 1B, 1C, 1D, and 1E show cMyBP-C organization. FIG. 1A shows the sarcomere spans from Z-disc to Z-disc with the A-band containing thick filaments and the I-band containing actin filaments. Force is generated by myosin and actin in the thin/thick filament overlap portion of the A-band. cMyBP-C molecules (vertical stripes) are present in the C-zones toward the center of the A-band. The C-zones overlap with thin filaments (except at very long sarcomere lengths). FIG. 1B shows full-length cMyBP-C domains C0 through C10. Ig-like domains are shown as circles and fibronectin type-Ill domains are shown as hexagons. FIG. 1C shows N-terminal domains C0 through C2 (C0-C2), containing the proline/alanine-rich linker (P/A) and the M-domain (M) that contains phosphorylation sites (P). FIG. 1D shows a C1 domain structure (PDB: 2AVG) with sites of the Cys probe mutations tested labeled. FIG. 1E shows M-domain structure (based on PDB: 2LHU) with sites of the Cys probe mutations tested labeled and PKA-mediated phosphorylation sites (bolded P's).

FIGS. 2A, 2B, and 2C show fluorescence lifetime decay for TR-FRET of the C0-C2 biosensor. FIG. 2A shows normalized TR-FRET waveform decays of 5 μM of C0-C2^{H225C.P330C} biosensor. The instrument response function (IRF) of water scattering is shown in grey. Donor-only (D) samples exhibit a fluorescence waveform decay and TR-FRET is observed in donor-acceptor (D-A) sample waveform decay. FIG. 2B shows the effect of PKA treatment to phosphorylate C0-C2 biosensor on donor-acceptor waveform decay as compared to unphosphorylated C0-C2. Region of fluorescence lifetime, where T decays to 1/e (˜37%), is highlighted by a dotted box. FIG. 2C shows this region is magnified from the dotted box region in FIG. 2B and shows a clear separation between D-A lifetime for − and +PKA. The actual lifetime is ˜5 ns less than shown on the x-axis due to the rising phase of decay from 0 to ˜5 ns (e.g., T_{DA} of ˜16.5 ns is measured as ˜11.5 ns).

FIGS. 3A, 3B, and 3C show PKA-mediated phosphorylation effects on the C0-C2 biosensor. FIG. 3A shows Donor-only (D) and Donor plus Acceptor (D-A) without or with PKA phosphorylation (+PKA) lifetimes of C0-C2^{Cys225.Cys330} biosensor derived from waveforms like those in FIG. 2. FIG. 3B shows the percent change (%) in lifetimes of D and D-A samples upon PKA treatment. FIG. 3C shows the change in FRET Efficiency (%) of D-A samples without and with PKA-mediated phosphorylation of C0-C2 biosensor. Data shown as mean±S.E., n=17-18. For each panel comparing with and without PKA, *p<0.003, **p<0.0001 (Student's t-test).

FIG. 4 shows the Z′-Factor test comparing unphosphorylated and phosphorylated C0-C2 biosensor lifetimes. Lifetimes of 5 mM D-A labeled C0-C2 biosensor without (red) or with PKA treatment (purple) in ˜40 wells each of a 384-well plate. In this test, each +PKA well was treated independently with PKA. Horizontal solid lines indicate 3×S.D. of the mean lifetimes (dotted line). Z′-factor is defined as the difference between 3×S.D. (A) divided by the difference in the mean signal (B) (Eq. 4). In this test, the Z′-factor is 0.60.

FIGS. 5A, 5B, 5C, and 5D show the effects of PKA-mediated phosphorylation on structural dynamics of the C0-C2 biosensor. Global analysis of TR-FRET waveforms using non-linear least squares regression. FIG. 5A shows a one-Gaussian probe to probe distance distributions of the C0-C2 biosensor without and with PKA treatment. Each population possesses a center distance (peak) and measure of disorder (FHWM) in angstroms (Å). FIG. 5B shows the center distances of the C0-C2 biosensor without and with PKA treatment. FIG. 5C shows FHWM of the C0-C2 biosensor without and with PKA treatment. FIG. 5D shows a cartoon of changes in structure show distance (length of double-sided arrows) and dynamics (arrow thickness and faded protein structure with the number of *'s representing disorder) of C0-C2 biosensor. For FIGS. 5A and 5B, data shown as mean S.E., n=4, and *p<0.003 and **p<0.0001 (Student's t-test).

FIGS. 6A, 6B, and 6C show the C0-C2 biosensor retains wild-type secondary structure, phosphorylation, and phosphorylation-sensitive actin-binding. FIG. 6A shows a CD spectroscopy showing the wavelength dependence of molar residue ellipticity (MRE) values of wild type and C0-C2^{Cys225.Cys330} biosensor were similar, indicating normal protein folding in the biosensor. FIG. 6B shows maximal phosphorylation levels with 7.5 ng PKA/μg of wild type and C0-C2^{Cys225.Cys330} biosensor by using Pro-Q Diamond/SYPRO staining intensity ratios was not significantly different, indicating that phosphorylation is normal in the biosensor. FIG. 6C shows actin cosedimentation assays for molar binding of unphosphorylated and phosphorylated wild type and C0-C2^{Cys225.Cys330} biosensor were similar. For all proteins and concentrations tested, PKA induced reduced C0-C2 binding, indicating a normal actin-binding function of the biosensor. Data shown as mean±S.E., n=6-9. For panel C, * denote statistical differences due to PKA phosphorylation of WT and biosensor for each respective C0-C2 concentration, *p<0.0001 (1-way ANOVA, Tukey's multiple comparisons).

### DETAILED DESCRIPTION OF THE INVENTION

Before the present compounds, compositions, and/or methods are disclosed and described, it is to be understood that this invention is not limited to specific synthetic methods or to specific compositions, as such may, of course, vary. It is also to be understood that the terminology used herein is for the purpose of describing particular embodiments only and is not intended to be limiting.

As used herein, the term “biosensor” refers to an analytical device, used for the detection of a chemical substance that combines a biological component with a physicochemical detector. The sensitive biological element, e.g. tissue, microorganisms, organelles, cell receptors, enzymes, antibodies, nucleic acids, etc., is a biologically derived material or biomimetic component that interacts with, binds with, or recognizes the analyte under study.

As used herein, the term “biosensor” may also refer to labeled-MyBP-C. In some embodiments, the MyBP-C may be labeled with one probe. In preferred embodiments, the MyBP-C is labeled with two probes. In some embodiments, the biosensor may be labeled at a positively charged loop within the C1 domain that interacts with tropomyosin and/or the tri-helix bundle region within the phosphorylatable M-domain. In some embodiments, the biosensor may be labeled within the C0-C2 domain of the MyBP-C protein. In some embodiments, the biosensor may comprise a mutated residue. In other embodiments, the biosensor protein comprises a mutated residue within the C0-C2 domain of the MyBP-C protein. In some embodiments, the biosensor may comprise a mutated cysteine residue. In other embodiments, the biosensor may comprise a mutated cysteine residue within the C0-C2 domain of a MyBP-C protein. In other embodiments, the biosensor may be labeled on a mutated residue.

As used herein, the term “fluorescent protein biosensor” refers to a biosensor comprising a protein and a fluorescent probe operably connected (i.e., a protein labeled with a fluorescent probe). As used herein, the term “fluorescent protein biosensor” may refer to a biosensor comprising a protein and two fluorescent probes operably connected (i.e., a protein labeled with two fluorescent probes) or may refer to a protein and one fluorescent probe and one non-fluorescent quencher operably connected.

As used herein, the term “high throughput” refers to the automation of experiments such that large-scale repetition becomes feasible.

As used herein, time-resolved fluorescence refers to time-resolved fluorescence spectroscopy is an extension of fluorescence spectroscopy. Here, the fluorescence of a sample is monitored as a function of time after excitation by a flash of light. The time resolution can be obtained in a number of ways, depending on the required sensitivity and time resolution. In preferred embodiments, direct waveform recording (DWR) is used. In other embodiments, time-correlated single photon counting (TCSPC) is used. In preferred embodiments, DWR or TCSPC is measured in an instrument with plate reader capabilities.

As used herein, fluorescent energy transfer (FRET) refers to a non-radiative transfer of energy between two nearby light-sensitive molecules (fluorophores). Here, a donor dye in an excited state can transfer a part of its energy to an acceptor molecule which then emits fluorescence at a specific wavelength. The emission from the acceptor can be detected as soon as both dyes are in close proximity. In some embodiments, when the two probes are in close proximity the donor emits less light (i.e. the donor emission is quenched when FRET occurs). In some embodiments, the donor and acceptor fluorescence emissions have different wavelengths that can be distinguished from each other.

As used herein, time-resolved fluorescent energy transfer (TR-FRET) refers to the combination of time-resolved (TR) measurement of fluorescence with fluorescence resonance energy transfer (FRET) technology.

As used herein, FRET-efficiency refers to the number of excited donors transferring energy to acceptors over (or divided by) the number of photons absorbed by the donor. FRET-efficiency may also refer to the proportion of the donor molecules that have transferred excitation state energy to the acceptor molecules, which increases with decreasing intermolecular distance

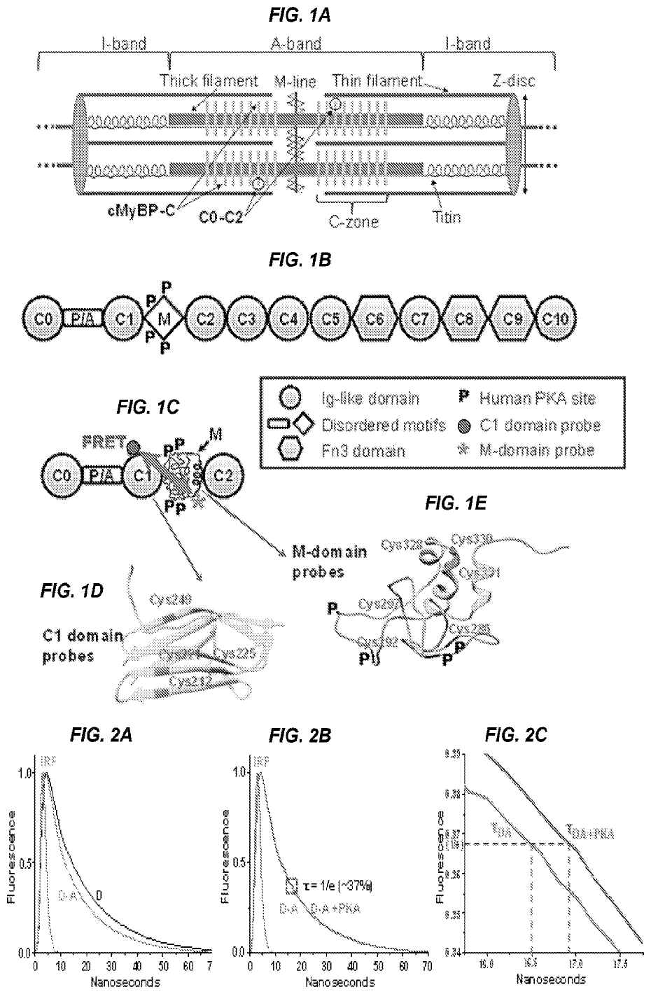

Referring now to FIGS. 1A-6C, the present invention features methods for using TR-FRET and a fluorescent protein biosensor to quantitate phosphorylation-mediated structural changes.

The present invention features a method of using time-resolved fluorescence energy transfer (TR-FRET) and a fluorescent protein biosensor to quantitate phosphorylation-mediated structural changes in solution. In some embodiments, the method comprises labeling (operably connecting) a protein with two fluorescent probes (or one fluorescent probe and one non-fluorescent quencher) for TR-FRET to generate a fluorescent protein biosensor suitable for TR-FRET. In other embodiments, the method comprises measuring FRET efficiency when structural changes in the fluorescent protein biosensor occur, wherein FRET efficiency is the proportion of the donor molecules that have transferred excitation state energy to the acceptor molecules. In further embodiments, the method comprises quantitating protein structural changes including phosphorylation using the measured FRET efficiency.

In other embodiments, the present invention may feature a method of using time-resolved fluorescence energy transfer (TR-FRET) to quantitate myosin binding protein-C (MyBP-C) phosphorylation-mediated structural changes in solution. In some embodiments, the method comprises labeling MyBP-C with two fluorescent probes suitable for TR-FRET. In some embodiments, the method comprises measuring FRET efficiency when structural changes in the MyBP-C occur, wherein FRET efficiency is the proportion of the donor molecules that have transferred excitation state energy to the acceptor molecules. In further embodiments, the method comprises quantitating MyBP-C structural changes including phosphorylation using the measured FRET efficiency. In some embodiments, a fluorescent probe suitable for time-resolved fluorescence energy transfer (TR-FRET) is any fluorescent dye described herein.

In some embodiments, the protein is labeled with two probes for TR-FRET (donor-acceptor). In other embodiments, the protein is labeled with one probe for TR-FRET (donor-only). In some embodiments, the protein is labeled with one fluorescent probe for TR-FRET. In some embodiments, the protein is labeled with two fluorescent probes for TR-FRET. In other embodiments, the protein is labeled with one fluorescent probe and one non-fluorescent quencher probe for TR-FRET.

In some embodiments, a solution may refer to a biochemical or physiological buffer. For example, 50 mM NaCl and 50 mM Tris, pH 7, or buffers with additional or alternative chemicals.

In some embodiments, a physiological change/perturbation, phosphorylation, and/or mutation of the fluorescent protein biosensor affects a change in the structure of the protein, affecting/changing FRET efficiency. Non-limiting examples of a physiological change and/or perturbation include but are not limited to binding to another protein (e.g., actin/thin filament or myosin/thick filament), temperature change, pH change, a change in ionic strength, or a combination thereof.

In some embodiments, the fluorescent protein biosensor may comprise a cardiac myosin binding protein-C (cMyBP-C), skeletal MyBP-C, and fragments thereof (e.g., C0-C2).

Structurally, cMyBP-C is a ˜140 kDa protein consisting of 8 Igl-like and 3 fibronectin-3 domains, referred to as C0-C10 (FIG. 1B). In some embodiments, the myosin binding protein-C (cMyBP-C) fragment may comprise the C0-C2 domains of the myosin binding protein. As used herein, “C0-C2,” “C0-C2 fragment,” or “C0-C2 domains” may be used interchangeable and refers to a MyBP-C N-terminal fragment that contains two flexible linker regions, the proline/alanine-rich linker (P/A) connecting C0 to C1 and the motif domain (M-domain) (FIG. 1C).

Non-limiting examples of quenchers may include but are not limited to DDPM, QSY-9, or a combination thereof. In some embodiments, the quenchers are dark quenchers. Non-limiting examples of the fluorescent probes comprise IAEDANS, IAANS, CPM, IANBD, FMAL, TMR, 5-IAF, ATTO, and Alexa Fluor dyes (including but not limited to Alexa Fluor 488, Alexa Fluor 532, and Alexa Fluor 568). In some embodiments, the fluorescent probes are covalently attached by thiol-reactive chemistry of the biosensor cysteines and each dye's maleimide or iodoacetamide group. In other embodiments, the fluorescent probes are thiol-reactive dyes. In other embodiments, the fluorescent probes comprise a 355-532 nm excitation range. Without wishing to limit the present invention to any theories or mechanisms it is believed that red-shifted dyes excited towards the 532 nm help reduce interference with compound autofluorescence in screens.

The present invention is not limited to thiol-reactive dyes covalently attached to cysteine residues, and may also include, for example, amine-reactive dyes covalently attached to an amine group.

In some embodiments, the method described herein is for screening physiological conditions or compounds that affect structural changes of the fluorescent protein biosensor.

In preferred embodiments, the labeling of a protein with a fluorescent probe is performed by industry-standard technology. The MyBP-C is reduced by equimolar TCEP (tris(2-carboxyethyl)phosphine) to cysteines. Donor-only dye is added to achieve ˜30% labeling of total cysteines. The reaction is stopped with excess DTT. Excess dye is removed. A portion (half or less) is saved as the donor-only sample and the other portion (half or more) is labeled at ˜70% of the remaining cysteines using acceptor dye and repeating the steps done for the donor dye. A key unique step is that the labeling reactions are done at mildly acidic pH 6.5 to remove non-specific labeling of additional residues, as non-specific labeling is detectable at higher pH 7, 7.5, and 8. Reproducibility of FRET data was improved by a hard clarification spin (100K×g for 30 min) of the final sample to remove any remaining dye or aggregates following exhaustive dialysis to ensure complete removal of remaining dye and/or protein aggregates.

Additionally, the present invention features a fluorescent biosensor protein suitable for time-resolved fluorescence energy transfer (TR-FRET). In some embodiments, the fluorescent biosensor protein may comprise two fluorescent probes operably connected to the protein (i.e., a protein labeled with two fluorescent probes). In other embodiments, the fluorescent biosensor protein is capable of quantitating phosphorylation-mediated structural changes in solution.

The present invention may also feature a method of using time-resolved fluorescence energy transfer (TR-FRET) to quantitate myosin binding protein-C (MyBP-C) phosphorylation-mediated structural changes in solution. In some embodiments, the method comprises labeling (operably connecting) a MyBP-C with two fluorescent probes for TR-FRET. In other embodiments, the method comprises measuring FRET efficiency when structural changes in the MyBP-C occur, wherein FRET efficiency is the proportion of the donor molecules that have transferred excitation state energy to the acceptor molecules.

In further embodiments, the method comprises quantitating protein structural changes including phosphorylation using the measured FRET efficiency.

In preferred embodiments, the MyBP-C is labeled with two probes for TR-FRET (donor-acceptor). In other embodiments, the MyBP-C is labeled with one probe for TR-FRET (donor). In some embodiments, the MyBP-C is labeled with one fluorescent probe for TR-FRET. In some embodiments, the MyBP-C is labeled with two fluorescent probes for TR-FRET. In other embodiments, the MyBP-C is labeled with one fluorescent probe and one non-fluorescent quencher probe.

In some embodiments, a physiological change/perturbation, phosphorylation, and/or mutation of the MyBP-C affects a change in structure of the protein, affecting/changing FRET efficiency.

In some embodiments, the myosin binding protein C (MyBP-C) comprises a cardiac myosin binding protein-C (cMyBP-C), skeletal MyBP-C, and fragments thereof (e.g., C0-C2).

Non-limiting examples of the fluorescent probe include IAEDANS, IAANS, CPM, IANBD, FMAL, TMR, 5-IAF, ATTO, and Alexa Fluor dyes (including but not limited to Alexa Fluor 488, Alexa Fluor 532, and Alexa Fluor 568). In some embodiments, the fluorescent probes are covalently attached by thiol-reactive chemistry of the biosensor cysteines and each dye's maleimide or iodoacetamide group. In other embodiments, the fluorescent probes are thiol-reactive dyes.

In further embodiments, the method described herein is for screening physiological conditions or compounds that affect structural changes of the MyBP-C.

In some embodiments, the labeling of the MyBP-C protein with a fluorescent probe is performed by industry-standard technology. The MyBP-C is reduced by equimolar TCEP to cysteines. Donor-only dye is added to achieve ˜30% labeling of total cysteines. The reaction is stopped with excess DTT. Excess dye is removed. A portion (half or less) is saved as the donor-only sample and the other portion (half or more) is labeled at ˜70% of the remaining cysteines using acceptor dye and repeating the steps done for the donor dye. A key unique step is that the labeling reactions are done at mildly acidic pH 6.5 to remove non-specific labeling of additional residues, as non-specific labeling is detectable at higher pH 7, 7.5, and 8. Reproducibility of FRET data was improved by a hard clarification spin (100K×g for 30 min) of the final sample to remove any remaining dye or aggregates following exhaustive dialysis to ensure complete removal of remaining dye and/or protein aggregates.

In some embodiments, the present method is screening physiological conditions or compounds that affect structural changes of the MyBP-C.

The present invention also features a fluorescent biosensor protein suitable for time-resolved fluorescence energy transfer (TR-FRET). In some embodiments, the fluorescent biosensor protein comprises a protein labeled with two fluorescent probes. In other embodiments, the fluorescent biosensor protein is capable of quantitating phosphorylation-mediated structural changes in solution.

In preferred embodiments, the present invention may also feature a myosin-binding protein C (MyBP-C) fluorescent biosensor suitable for time-resolved fluorescence energy transfer (TR-FRET). In some embodiments, the MyBP-C fluorescent biosensor protein comprises a MyBP-C labeled with two fluorescent probes The MyBP-C fluorescent biosensor protein may be capable of quantitating phosphorylation-mediated structural changes in solution.

In some embodiments, the present invention features a fluorescent biosensor protein suitable for time-resolved fluorescence energy transfer (TR-FRET). In some embodiments, the fluorescent biosensor protein (e.g., MyBP-C) comprises a protein (e.g., MyBP-C) labeled with at least one probe. In other embodiments, In some embodiments, the fluorescent biosensor protein (e.g., MyBP-C) comprises a protein (e.g., MyBP-C) labeled with at least two probes. In some embodiments, the fluorescent biosensor protein is capable of quantitating phosphorylation-mediated structural changes in solution. In some embodiments, the protein may be labeled with a fluorescent probe, a non-fluorescent quencher, or a combination thereof.

In other embodiments, the present invention features a myosin-binding protein C (MyBP-C) fluorescent biosensor protein suitable for time-resolved fluorescence energy transfer (TR-FRET). In some embodiments, the MyBP-C fluorescent biosensor protein comprises a MyBP-C labeled with at least one fluorescent probe. In other embodiments, the MyBP-C fluorescent biosensor protein comprises a MyBP-C labeled with at least two fluorescent probes. In further embodiments, the MyBP-C fluorescent biosensor protein may be labeled with a fluorescent probe, a non-fluorescent quencher, or a combination thereof. In some embodiments, the MyBP-C fluorescent biosensor protein is capable of quantitating phosphorylation-mediated structural changes in solution.

In some embodiments, the present invention features methods for using time-resolved fluorescence energy transfer (TR-FRET) to quantitate phosphorylation-mediated structural changes in solution utilizing any one of the fluorescent protein (e.g., MyBP-C) biosensors as described herein.

In some embodiments, the fluorescent protein biosensor can be used with compound libraries to identify hits. In other embodiments, the assay/methods described herein are sensitive to detecting compounds that bind to cMyBP-C and mimic phosphorylated or dephosphorylated states, which modulates binding to actin/myosin and thereby enhances cardiac muscle function as new therapy for heart failure.

Furthermore, in some embodiments, the assay/methods described herein are sensitive to disease mutations, making this assay useful for testing the pathogenicity of newly identified mutations in cMyBP-C in the clinic (two-week turn-around from identifying mutation to results, especially in this N terminal region of the molecule). In some embodiments, the disease mutation is nearby the phosphorylation on the probes.

### Example

The following is a non-limiting example of the present invention. It is to be understood that said example is not intended to limit the present invention in any way. Equivalents or substitutes are within the scope of the present invention.

Recombinant human cMyBP-C. A pET45b vector encoding E. coli optimized codons for the C0-C2 fragment of human cMyBP-C with N-terminal 6×His tag and TEV protease cleavage site was obtained from GenScript (Piscataway, NJ). C0-C2 biosensor mutants were engineered using a Q5 Site-Directed Mutagenesis Kit (New England BioLabs, Ipswich, MA). Substitution mutations were performed to remove endogenous cysteines to generate C0-C2^{Cys249} and C0-C2^{Cys-free} constructs. C0-C2^{Cys249} was generated by performing the following mutations: C239L, C426T, C436V, and C443S. C0-C2^{Cys-free} was generated by performing the following mutations: C239L, C249S, C426T, C436V, and C443S. Using the C0-C2^{Cys249} template, the following mutations were introduced to generate di-Cys C0-C2 biosensors: S286C, 1292C, S297C, A328C, P330C, and S331C. These are referred to as C0-C2^{Cys249.Cys286}, C0-C2^{Cys249.Cys292}, C0-C2^{Cys249.Cys297}, C0-C2^{Cys249.Cys328}, C0-C2^{Cys249.Cys330}, and C0-C2^{Cys249.Cys331}, respectively. Using the C0-C2^{Cys-free} template, the following mutations were introduced to generate mono-Cys C0-C2: S212C, L221C, and H225C. These are referred to as C0-C2^{Cys212}, C0-C2^{Cys221} and C0-C2^{Cys225}, respectively. An additional cysteine was substituted into the mono-Cys C0-C2 to generate di-Cys C0-C2 biosensors (S297C and P330C) referred to as C0-C2^{Cys212.Cys297}, C0-C2^{Cys212.Cys330}, C0-C2^{Cys221.Cys330}, C0-C2^{Cys225.Cys297} and C0-C2^{Cys212.Cys330}. All sequences were confirmed by DNA sequencing (Eton Biosciences, San Diego, CA).

Protein production in E. coli BL21(DE3) competent cells (New England Bio Labs, Ipswich, MA) and purification of C0-C2 proteins using His60 Ni Superflow resin was done as previously described. The His-tag was removed by TEV protease digestion and C0-C2 were concentrated, dialyzed in 50/50 buffer (50 mM NaCl and 50 mM Tris, pH 6.5 or 7.5) and stored at 4° C. Initial screening of C0-C2 biosensors was performed in a 50/50 buffer at pH 7.5. C0-C2^{Cys225.Cys330} was additionally purified using size exclusion chromatography to achieve >90% intact C0-C2. For size exclusion chromatography C0-C2 (10-15 mg/ml in 3.5-4.5 ml) was applied in running buffer (150 mM NaCl, 50 mM NaPO₄, 1 mM DTT, pH 6.7) to a HiPrep Sephacryl S-100 column. Flow rate was 0.7 ml/min. Purified C0-C2 was checked for purity by SDS-PAGE and then dialyzed into the appropriate buffer, usually 50/50 for labeling. At least two protein preparations were performed for each experiment and proteins were typically used within two weeks of purification.

Labeling of human C0-C2. C0-C2 labeling reactions were performed in 50/50 buffer in dim-lighting conditions. For donor and acceptor labeling, 60 μM of 5-((((2-Iodoacetyl)amino)ethyl)amino)Naphthalene-1-Sulfonic Acid] (IAEDANS) and 200 μM of N-[4-(dimethylamino)-3,5-dinitrophenyl]maleimide (DDPM) in 50/50 buffer at pH 6.5 containing 50 μM C0-C2. For initial biosensor screening studies, C0-C2 was labeled at pH 7.5. For labeling, 50 μM C0-C2 was reduced using 0.2 mM tris(2-carboxyethyl)phosphine (TCEP) for 30 min at 25° C. while rocking. Dye stocks were thawed (stored at ˜80° C. in DMF) and added to C0-C2 while slowly vortexing. C0-C2 was labeled for 1 hour at 25° C., while rocking. After labeling, dithiothreitol (DTT) was added in 5×molar excess of dye concentration to terminate the reaction of unbound dye. Labeled C0-C2 was dialyzed against 50/50 buffer at 100×volume excess with two buffer changes to remove remaining dye and DTT. To remove any precipitated dye and insoluble C0-C2, donor-acceptor labeled C0-C2 was centrifuged for 30 min at 100,000 RPM (350,000×g) in a Beckman TLA-120.2 rotor. The extent of protein labeling was determined by measuring dye absorbance at its maximal excitation wavelength using UV-Vis spectroscopy and dividing by the dye's extinction coefficient. Protein concentration was determined by Pierce™ BCA Protein Assay Kit (Thermo Fisher Scientific, Waltham, MA) as recommended by the supplier. Percent labeling was calculated by dividing the molar concentration of dye by the molar concentration of C0-C2 cysteines (50 μM of C0-C2 containing two cysteines is 100 μM of cysteines).

In vitro phosphorylation of cMyBP-C. For quantifying protein kinase A (PKA)-mediated phosphorylation of cMyBP-C, Pro-Q Diamond phosphoprotein stain (ThermoFischer, Waltham, MA) was used to visualize phosphorylated C0-C2 and SYPRO Ruby stain (ThermoFischer, Waltham, MA) was used for determining total protein according to the supplier's instructions. C0-C2 was dialyzed into either 50/50 buffer with 0.2 mM ATP, 1 mM DTT, and 2 mM MgCl2 or MOPS-actin binding buffer (M-ABB; 100 mM KCl, 10 mM MOPS pH 6.8, 2 mM MgCl2, 0.2 mM CaCl2, and 1 mM sodium azide) with 0.2 mM ATP, 1 mM DTT. C0-C2 was treated with 7.5 ng PKA/μg C0-C2 for 30 min at 30° C. Maximal phosphorylation is achieved at 2.5 ng PKA/μg C0-C2.

Actin preparations. Actin was prepared from rabbit skeletal muscle by extracting acetone powder in cold H₂O. The day prior to actin binding experiments (cosedimentation), G-actin was polymerized by the addition of MgCl₂ to a final concentration of 3 mM for 1 hour at 25° C. F-actin was collected by centrifugation at 4° C., 100,000 RPM (350,000×g) in a Beckman TLA-120.2 rotor and the pellet was resuspended in M-ABB. Any bundled actin was removed by centrifugation at 5,000×g, 40C for 5 min in an Eppendorf 5424R table-top microfuge.

Actin cosedimentation assays and binding analysis. Actin binding by cMyBP-C C0-C2 fragments was determined by cosedimentation and analyzed. Actin binding levels were determined at 25° C. in M-ABB using 1 μM phalloidin-stabilized F-actin incubated with 1.25 or 2.5 μM C0-C2 for 30 minutes.

Circular dichroism. Circular dichroism (CD) spectroscopy of wild type and donor-acceptor labeled C0-C2^{Cys225.Cys330} constructs was performed to measure secondary structural content of C0-C2. CD spectra of 0.2 mg/ml of C0-C2 with a 0.5 mm pathlength cuvette were acquired on a DSM-20 CD spectrometer (Olis, Bogart, GA) in 20 mM sodium borate buffer (pH 7.0) with 1 mM DTT added one hour prior to readings at 25±1° C. Background signal was corrected by subtracting the spectra of the buffer from the C0-C2 spectra. Three repeated scans of spectral readings were acquired at 1-nm intervals from 195 to 250 nm. Ellipticity from wavelengths 210 to 240 nm (by 1-nm increments) were analyzed for secondary structure analysis using predictive algorithms of the K2D3 web-based software.

Time-Resolved FRET (TR-FRET) data acquisition and analysis. 50-μl sample aliquots were loaded manually in 384-well black polypropylene microplates (#781209, Greiner Bio-One, Monroe, NC). Plates were spun for 1 min at 1,000 RPM (˜200×g) in an Eppendorf centrifuge and rotor (5810R A-4-81) to remove air bubbles. Donor (IAEDANS)-labeled or donor-acceptor (IAEDANS-DDPM)-labeled C0-C2 (5 μM) was excited using a passively Q-switched microchip frequency-tripled Nd:YAG 355/532 nm combo laser (SNV-05P-100; Teem Photonics, Meylan, France) at 355 nm using a band-pass excitation filter (340126 nm) with a pulse repetition frequency of ˜8 kHz. Emitted photons were passed through a polarizer set at the magic angle 54.7°, followed by an interference band-pass filter (470/20 nm; Semrock, Rochester, NY), and detected with a photomultiplier tube module (H10720-210; Hamamatsu Photonics, Hamamatsu, Japan) and 1 GHz analog transient waveform digitizer (ATWD version 3.1) (Kleinfelder, Proc. SPIE 2003) in a Fluorescence Lifetime Plate Reader (FLTPR) instrument from Fluorescence Innovations (Minneapolis, MN). Fluorescence emission decays were acquired at 25° C. in 50/50 buffer with 0.2 mM ATP, 1 mM DTT, and 2 mM MgCl₂.

Fluorescence waveforms observed for each well of donor-only or donor-acceptor C0-C2 with or without PKA treatment were convolved with the instrument response function (IRF) to determine the lifetime (τ) (Eq. 1). Data to analyze lifetimes to calculate FRET efficiency were fit to one-exponential decays using MatLab, version 7.1; The MathWorks. TR-F waveforms for donor-only (D_{only}) and donor-acceptor (D-A) labeled C0-C2^{Cys225.Cys330} were analyzed globally to determine center distances between probes and widths of Gaussian distributions (full-width at half maximum, FWHM). Fluorescence waveforms for donor-only (D_{only}) and donor-acceptor (D-A) labeled C0-C2^{Cys225.Cys330} were analyzed using TR-FRET analysis software. D_{only} waveforms were best-fit to three exponentials and D-A waveforms were best-fit to a one-Gaussian distance distribution determined by minimization of χ² values from the residual plots of the waveform fittings. The TR-FRET distance distribution was characterized by a center distance Rⱼ and a full width at half-maximum Γⱼ.

The decay of the excited state of the fluorescent IAEDANS donor dye attached to C0-C2 to the ground state is listed below in Equation 1:

$$I ( t ) = {I}_{0} exp ( - \frac{t}{τ} ) \qquad ( Eq . 1 )$$

where I₀ is the peak fluorescence intensity upon excitation (t=0) and τ is the fluorescence lifetime (t=τ when I decays to 1/e or ˜37% of I₀). FRET efficiency values were calculated by Equation 2 listed below:

$$FRET Efficiency ( \% ) = ( 1 \frac{{τ}_{DA}}{{τ}_{D}} ) \times 100 \qquad ( Eq . 2 )$$

where the T_{D} and T_{DA} represents lifetime of D_{only} and DA labeled C0-C2 biosensor, respectively. Percent change is calculated in Equation 3 listed below as a function of FRET efficiency (E) biosensor with and without PKA treatment:

$$Percent Change ( \% ) = ( \frac{{E}_{+ PKA} - {E}_{- PKA}}{{E}_{- PKA}} ) \times 100 \qquad ( Eq . 3 )$$

Determination of Z′-factor for C0-C2^{Cys225.Cys330}. For suitability in high-throughput screening (HTS), TR-FRET assay quality was determined for unphosphorylated versus phosphorylated donor-acceptor labeled C0-C2^{Cys225.Cys330}. Each phosphorylation was performed independently for each well. The lifetimes of unphosphorylated donor-acceptor labeled C0-C2^{Cys225.Cys330}. (T_{A}, n=37) and phosphorylated donor-acceptor labeled C0-C2^{Cys225.Cys330} (T_{B}, n=39) were compared and indexed by the Z′-factor:

$${Z}^{′} = 1 - \frac{3 ( {σ}_{A} + {σ}_{B} )}{\vert {μ}_{A} - {μ}_{B} \vert} \qquad ( Eq . 4 )$$

where σ_{A} and σ_{B} are the standard deviations (SD) of the T_{A} and T_{B} lifetimes, respectively, μ_{A} and μ_{B} are the means of the T_{A} and T_{B} lifetimes, respectively. Z′-factor of less than 0 is “useless”, 0 to 0.5 is “good” and 0.5 to 1.0 is “excellent” assay quality.

Modification of human C0-C2 for use in site-specific spectroscopy studies. To monitor structural dynamics in human cMyBP-C N-terminal regions (C0-C2, FIG. 1C), TR-FRET probes were attached to different regions of the C1 and M-domains (FIGS. 1D and 1E). Previous studies found that murine C0-C2 is labeled with thiol-reactive TR-FRET dyes only at one exposed cysteine, Cys248. The remaining four cysteines are buried in C1 and C2 and are not readily labeled. Initial labeling of human protein resulted in dye:C0-C2 ratios that were much greater than 1:1 (as high as 4:1 for some probes, data not shown). This suggested that some of the 4 cysteines thought to be buried in human C1 and C2 were reactive during labeling, in addition to Cys249 (the human residue homologous to the mouse Cys248). Therefore, these 4 cysteines were substituted by site-directed mutagenesis leaving only the surface exposed Cys249 (referred to as C0-C2^{Cys249}). To probe other regions of C1 besides Cys249, all 5 endogenous cysteines were removed (referred to as C0-C2^{Cys-free}).

Designing and testing of di-Cys C0-C2 FRET biosensors for detecting phosphorylation-dependent structural changes. TR-FRET between probes on C1 and C2 in mouse C0-C2 showed structural changes upon PKA phosphorylation. Introduction of cysteines at multiple sites in C2 of human C0-C2^{Cys249} resulted in destabilized and degraded protein being produced in bacteria (data not shown). Therefore, C0-C2^{Cys249} and C0-C2^{Cys-free} were used as templates to substitute additional cysteine(s) in the C1 and M-domains. Each potential FRET-based biosensor construct is a di-Cys C0-C2 protein designed to examine phosphorylation-sensitive structural dynamics between C1 and the M-domain (FIG. 1C, 1D, and 1E). Table 1 shows the percent change in FRET efficiency that resulted from PKA treatment of each construct.

TABLE 1
Potential human C0-C2 biosensors: PKA-mediated phosphorylation
effects on FRET efficiency

| | Percent change in | Number | Number |
|---|---|---|---|
| Cysteine Location | FRET | of PKA | of Protein |
| (C1 Domain.M-domain) | Efficiency | Treatments | Preparations |
| Cys249.Cys286 | −0.8 + 0.1 | 5 | 2 |
| Cys249.Cys292 | −2.3 + 1 | 9 | 4 |
| Cys249.Cys297 | 2.9 + 0.4 | 4 | 2 |
| Cys249.Cys328 | −1.3 + 0.5 | 7 | 3 |
| Cys249.Cys330 | −5.4 + 0.6 | 7 | 3 |
| Cys249.Cys331 | −3.4 + 0.5 | 7 | 3 |
| Cys212.Cys297 | −7.8 + 1.5 | 5 | 2 |
| Cys225.Cys297 | −5.1 + 0.9 | 6 | 2 |
| Cys212.Cys330 | −11.8 + 0.6 | 5 | 2 |
| Cys221.Cys330 | −17.2 + 0.7 | 10 | 3 |
| Cys225.Cys330 | −18.2 + 0.6 | 6 | 2 |
| Cys225.Cys330 | −16.7 + 0.3 | 17 | 4 |
| (pH 6.5) | | | |

Primary sequence location of cysteines in C1 and M-domain of each human di-Cys C0-C2 biosensor tested.
FRET efficiency is calculated using Eq. 2 from donor and donor-acceptor lifetimes for each biosensor ± phosphorylation.
Percent change in FRET efficiency due to phosphorylation is calculated using Eq. 3 and shown as mean ± SE, n = 4-17.
The number of replicates for individual phosphorylation treatments (technical repeats) and protein preparations (biological repeats) are shown in columns on the right side.

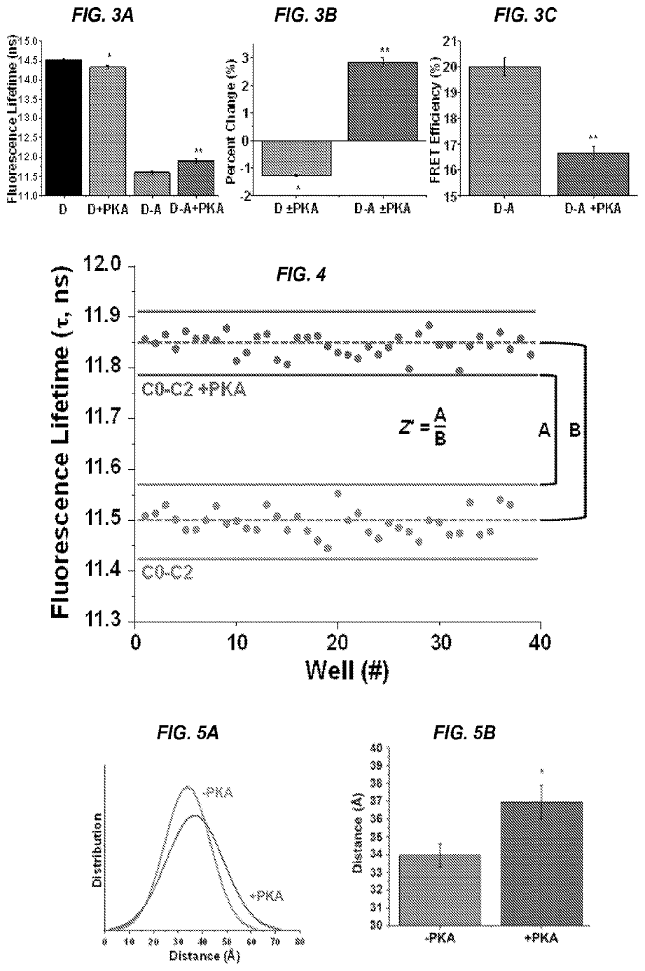

Six constructs were tested using the C1 Cys249 paired with a second cysteine at positions within the phosphoserine region (positions 286, 292, or 297) or in the tri-helix bundle region (positions 328, 330, or 331) of the M-domain. C0-C2^{Cys249.Cys297} is the only construct with a PKA-mediated increase in FRET efficiency at ˜2.9% while C0-C2^{Cys249.Cys330} reported the largest reduction with a ˜5.4% decrease in FRET efficiency. Next, structural changes with C1 probes placed on the other side of the Ig-like domain relative to Cys249 were surveyed. For this C0-C2^{Cys212}, C0-C2^{Cys221}, and C0-C2^{Cys225} constructs were generated (FIG. 1D). These were paired with M-domain cysteines at either Cys297 or Cys330, as these locations reported the largest changes in C0-C2^{Cys249} constructs. C0-C2^{Cys249.Cys330} exhibited the largest PKA-mediated changes in FRET efficiency (further described in FIGS. 3A, 3B, and 3C) and was selected for further characterization.

Fluorescence waveforms of IAEDANS labeled C0-C2^{Cys225.Cys330} (D_{only}) and IAEDANS plus DDPM labeled C0-C2^{Cys225.Cys330} (D-A) are shown in FIG. 2A. The presence of the acceptor (D-A) causes a left-ward shift of the waveform (relative to D_{only}) due to FRET. The effect of phosphorylation is observable on the D-A waveform causing a right-ward shift indicating less FRET (FIGS. 2B and 2C). Fluorescence lifetime of D_{only} and D-A samples derived from waveforms are shown in FIGS. 3A, 3B and 3C and FRET efficiencies were calculated from these lifetimes. FRET reduced lifetime in D-A samples (11.60±0.05 ns) compared to D_{only} samples (14.52±0.04 ns) (FIG. 3A). PKA treatment of D-A C0-C2 increases lifetime by 2.84% (FIGS. 3A and 3B) and significantly reduces FRET efficiency from 20.00±0.34% to 16.66±0.28% (p<0.0001) (FIG. 3C), indicating a 16.65±0.33% reduction in FRET efficiency (Table 1). Interestingly, a small (1.28%) but significant reduction in lifetime of IAEDANS upon PKA treatment is observed for D_{only}, C0-C2^{Cys225.Cys330}, suggesting small but detectable effects of phosphorylation on the probe environment.

Evaluation of cMyBP-C biosensor. C0-C2^{Cys225.Cys330}, suitability for high-throughput screening (HTS). C0-C2^{Cys225.Cys330} suitability as a sensor was tested in mock screens for small-molecules drugs that bind to unphosphorylated C0-C2 and induce N-terminal structural changes that recapitulated phosphorylated structural dynamics by deterring a Z′ factor. Lifetime averages and standard deviations (S.D.) of the donor probe (IAEDANS) in donor-acceptor (IAEDANS-DDPM) labeled C0-C2^{Cys225.Cys330} were determined for non-phosphorylated and phosphorylated C0-C2^{Cys225.Cys330}. These values were then used to calculate the Z′-factor (Eq. 4). Z′ factors less than 0 indicate that screens done under the conditions tested would be useless. Z′-factors between 0.0 and 0.5 indicate a doable screen, and Z′ factors between 0.5 and 1.0 are indicative of an excellent screen. Using a 384-well plate, DA C0-C2^{Cys225.Cys330} was tested for PKA-mediated changes in lifetime. FIG. 4 shows 76 wells of D-A biosensor plotted as a function of IAEDANS lifetime. The average lifetimes of unphosphorylated and phosphorylated C0-C2^{Cys225.Cys330} are 11.50±0.025 ns and 11.85±0.022 ns, respectively, (FIG. 4). This difference of 3.04% is highly significant (p<0.0001). Z′-factor for this comparison is 0.60, which classifies the assay as “excellent” for use in HTS. Furthermore, this was repeated with 4 independent protein preparations and in each case Z′ values were in the excellent range (Table 2).

TABLE 2
Z′ Factor Score for each Protein Preparation of D-A C0-C2^{Cys225.Cys330} ± PKA.

| | | D-A | D-A + PKA | Lifetime | | |
|---|---|---|---|---|---|---|
| (#) | Number of | Lifetime | Lifetime | Difference | Sum of 3 × | |
| Preparation | wells (n) | (ns) | (ns) | (ns) | S.D. | Z′ Factor |
| Prep. 1 | 28 | 11.71 | 12.00 | 0.29 | 0.10 | 0.67 |
| Prep. 2 | 20 | 11.61 | 11.96 | 0.36 | 0.08 | 0.77 |
| Prep. 3 | 18 | 11.64 | 11.95 | 0.31 | 0.09 | 0.70 |
| Prep. 4 | 20 | 11.17 | 11.58 | 0.40 | 0.09 | 0.78 |
| Prep. 5 | 76 | 11.50 | 11.85 | 0.35 | 0.14 | 0.60 |
| (FIG. 4) | | | | | | |

Evaluating the Z′ Factor for each protein preparation of donor-acceptor labeled C0-C2^{Cys225.Cys330}. Mean nanosecond (ns) lifetimes and lifetime differences were calculated from each replicate within its respective protein preparation. The same approach calculated 3× standard deviations (3 × SD) from unphosphorylated and phosphorylated lifetimes with summation of each value shown as “Total Sum of 3 × SD”. Z′ Factor is calculated using Eq. 4 from the values listed for its preparation number. For Preparation 5, phosphorylation was done separately for each of 37 wells. For Preparations 1-4, phosphorylation was done in bulk batch and aliquoted into different wells. See FIG. 4 for the representative example Z′ Factor of C0-C2^{Cys225.Cys330}.

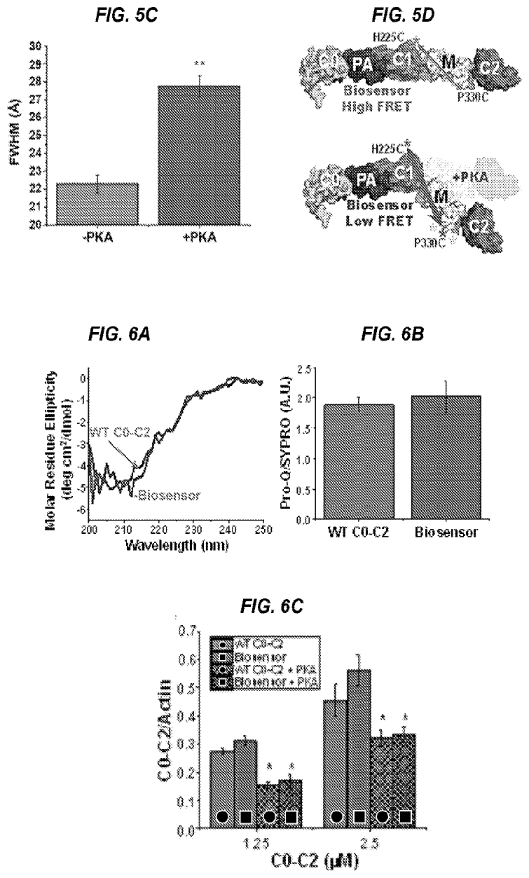

Evaluating the effects of PKA-mediated phosphorylation on C0-C2^{Cys225.Cys330} structural dynamics. TR-FRET waveforms were analyzed globally for C0-C2^{Cys225.Cys330} to determine the effect of phosphorylation on the center distance (Rⱼ) and the distribution of distances, expressed as FWHM (Γⱼ), between the probes, which is an indicator of molecular disorder. Both unphosphorylated and phosphorylated IAEDANS-DDPM labeled C0-C2^{Cys225.Cys330} were best-fit to a one-Gaussian distance distribution and shown in FIG. 5A. C0-C2 phosphorylation causes a rightward-shift and wider distribution of the structural population indicating increases in Rⱼ and Γⱼ. FIG. 5B shows phosphorylation significantly increases Rⱼ by 8.8% from 33.98±0.07 Å to 36.95±0.09 Å (p<0.05), a ΔRⱼ of 2.98 Å. FIG. 5C shows phosphorylation significantly increases Γⱼ by 24.4% from 22.30±0.05 Å to 27.75±0.06 Å (p=0.0004), a ΔΓⱼ of 5.45 Å. FIG. 5D shows a C0-C2 cartoon illustrating these changes to C0-C2 structure.

C0-C2^{Cys225.Cys330} retains wild type secondary structure. Circular dichroism (CD) spectroscopy was employed to quantify the secondary structural content (i.e., α-helix, β-sheet, and turns/undefined) of wild type C0-C2 and labeled C0-C2^{Cys225.Cys330}. The molar residue ellipticity values plotted against wavelength were similar for both C0-C2 constructs (FIG. 6A). They displayed a single, broad negative band in the far-UV wavelength region (200-220 nm) permitting well-defined calculation of secondary structure content. For wild type and biosensor (i.e, C0-C2^{Cys225.Cys330}) C0-C2, α-helical content was estimated to be, 2.3% and 2.8%, β-sheet content was estimated to be 38.6% and 38.5%, and turns/undefined to be 59.1% and 58.8%, respectively.

C0-C2^{Cys225.Cys330} exhibits normal sensitivity to PKA-mediated phosphorylation. PKA-mediated phosphorylation of wild type C0-C2 and C0-C2^{Cys225.Cys330} (i.e., the biosensor) was compared using Pro-Q Diamond phospho-staining and SYPRO Ruby total protein staining. Using standard conditions, no difference was found in phosphorylation levels (FIG. 6B).

C0-C2^{Cys225.Cys330} maintains cMyBP-C C0-C2 actin-binding function. C0-C2^{Cys225.Cys330} and wild type C0-C2 were evaluated for actin binding by using a filamentous actin (F-actin) cosedimentation assays. Wild type C0-C2 binding increases in a concentration-dependent manner and is significantly reduced by PKA phosphorylation. The most useful comparisons are found at submaximal binding levels (MyBP-C:actin; 1:7) found in cardiac muscle. C0-C2^{Cys225.Cys330} binding to actin was concentration- and PKA phosphorylation-dependent and similar to that observed for wild type C0-C2 (FIG. 6C).

As used herein, the term “about” refers to plus or minus 10% of the referenced number.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting essentially of” or “consisting of”, and as such the written description requirement for claiming one or more embodiments of the present invention using the phrase “consisting essentially of” or “consisting of” is met.

1. A method of using time-resolved fluorescence energy transfer (TR-FRET) and a fluorescent protein biosensor to quantitate phosphorylation-mediated structural changes in solution, the method comprising:
a. labeling a myosin-bindinq protein C (MyBP-C) with two fluorescent probes to generate a MyBP-C fluorescent protein biosensor suitable for TR-FRET;
b. measuring FRET efficiency when structural changes in the MyBP-C fluorescent protein biosensor occur, wherein FRET efficiency is a proportion of donor molecules that have transferred excitation state energy to acceptor molecules; and
c. quantitating MyBP-C fluorescent protein biosensor structural changes including phosphorylation using the measured FRET efficiency.

2. The method of claim 1, wherein a physiological change/perturbation, phosphorylation, and/or mutation of the fluorescent protein biosensor affects a change in structure of the protein, affecting/changing FRET efficiency, wherein FRET efficiency is the proportion of the donor molecules that have transferred excitation state energy to the acceptor molecules.

3. The method of claim 1, wherein the MyBP-C comprises a cardiac myosin binding protein-C (cMyBP-C), skeletal MyBP-C, or fragments thereof.

4. The method of claim 3, wherein the fragment of the protein comprises a C0-C2 fragment of a MyBP-C.

5. The method of claim 1, wherein the fluorescent probe is selected from a group consisting of IAEDANS, IAANS, CPM, IANBD, 5-IAF, TMP, ATTO FMAL, Alexa Fluor 488, Alexa Fluor 532, and Alexa Fluor 568.

6. The method of claim 1, wherein the method is for screening physiological conditions or compounds that affect structural changes of the fluorescent protein biosensor.

7. A method of using time-resolved fluorescence energy transfer (TR-FRET) and a fluorescent protein biosensor to quantitate phosphorylation-mediated structural changes in solution, the method comprising:
a. labeling a human myosin-binding protein C (MyBP-C) with two fluorescent probes to generate a MyBP-C fluorescent protein biosensor suitable for TR-FRET;
b. measuring FRET efficiency when structural changes in the MyBP-C fluorescent protein biosensor occur, wherein FRET efficiency is the proportion of the donor molecules that have transferred excitation state energy to the acceptor molecules; and
c. quantitating the MyBP-C fluorescent protein biosensor structural changes including phosphorylation using the measured FRET efficiency.

8. The method of claim 7, wherein a physiological change/perturbation, phosphorylation, and/or mutation of the MyBP-C affects a change in structure of the protein, affecting/changing FRET efficiency, wherein FRET efficiency is the proportion of the donor molecules that have transferred excitation state energy to the acceptor molecules.

9. The method of claim 7, wherein the human MyBP-C comprises a cardiac myosin binding protein-C (cMyBP-C), skeletal MyBP-C, and fragments thereof.

10. The method of claim 9, wherein the fragment of the MyBP-C comprises the C0-C2 fragment of the MyBP-C.

11. The method of claim 7, wherein the fluorescent probe is selected from a group consisting of IAEDANS, IAANS, CPM, IANBD, 5-IAF, TMP, ATTO FMAL, Alexa Fluor 488, Alexa Fluor 532, and Alexa Fluor 568.

12. The method of claim 7, wherein the method is for screening physiological conditions or compounds that affect structural changes of the MyBP-C.

13. A myosin-bindinq protein C (MyBP-C) fluorescent biosensor protein suitable for time-resolved fluorescence energy transfer (TR-FRET), the MyBP-C fluorescent biosensor protein comprises of a MyBP-C labeled with two fluorescent probes, wherein the MyBP-C fluorescent biosensor protein is capable of quantitating phosphorylation-mediated structural changes in solution.

14. The biosensor of claim 13, wherein the MyBP-C comprises a human MyBP-C, wherein the human MyBP-C comprises a human cardiac myosin binding protein-C (cMyBP-C), a human skeletal MyBP-C, and fragments thereof.

15. The method of claim 14, wherein the fragment of the human MyBP-C comprises a C0-C2 fragment of the human MyBP-C.

16. The biosensor of claim 13, wherein the fluorescent probe is selected from a group consisting of IAEDANS, IAANS, CPM, IANBD, 5-IAF, TMP, ATTO FMAL, Alexa Fluor 488, Alexa Fluor 532, and Alexa Fluor 568.

17.-27. (canceled)
